(19) United States
(12) Patent Application Publication
(10) Pub. No.: US 2010/0271592 A1
(43) Pub. Date: Oct. 28, 2010

(54) APPARATUS FOR MEASURING CRITICAL FLICKER FUSION FREQUENCY AND METHODS OF USING SAME

(75) Inventors: Bradley J. Katz, Salt Lake City, UT (US); Paul R. Borgmeier, Salt Lake City, UT (US)

Correspondence Address: Ballard Spahr LLP, SUITE 1000, 999 PEACHTREE STREET, ATLANTA, GA, 30309-3915, US

(73) Assignee: University of Utah Research Foundation, Salt Lake City, UT (US)

(21) Appl. No.: 12/525,768

(22) PCT Filed: Feb. 1, 2008
(86) PCT No.: PCT/US08/52832

§ 371 Date: May 19, 2010

Related U.S. Application Data
(60) Provisional application No. 60/888,882, filed on Feb. 8, 2007.

(51) Int. Cl. A61B 3/08

(52) U.S. Cl. 351/201

(57) ABSTRACT

Apparatuses and methods are provided for measuring the critical flicker fusion frequency of a subject. The apparatus in one aspect comprises two or more light sources, each flickering at a different predetermined frequency. The subject selects one light source from an array of light sources defining a first range of frequencies, the selection representing the light source of lowest frequency that appears as fused to the subject. The frequencies of the light sources are adjusted to second predetermined frequencies defining a second range. The subject selects the light source of lowest frequency that appears as fused to the subject. The frequency value of the second selection can be assigned as the subject's critical flicker fusion frequency.

### FIELD OF THE INVENTION

This invention relates generally to apparatuses and methods for measuring the critical flicker fusion frequency (CFFF) of a subject. More specifically, this invention relates to apparatuses and methods configured to display an array of lights to the subject, receive a selection of the light of lowest frequency that appears to the subject as a fused light, and assign the frequency value as the subject's CFFF.

### BACKGROUND OF THE INVENTION

As a light blinks faster and faster, at some frequency the blinking light will appear as a fused or continuous light to a subject. The frequency at which this phenomenon occurs is known as the critical flicker fusion frequency (CFFF). Critical flicker fusion frequency is affected by the size of the stimulus, color and brightness of the stimulus, duty cycle, and ambient light. CFFF is also an index of optic nerve function. Diseases that affect the integrity of the optic nerve decrease the CFFF of the affected subject. These diseases include optic neuritis, multiple sclerosis, tumors of the optic nerve (such as meningioma), and Graves' disease. As an example, a normal (i.e., unaffected) eye can be able to track a flickering light source up to 30 Hz. A diseased eye, such as an eye with optic neuritis, can view the same flickering light as fused at 10 Hz. As the disease process recovers, the CFFF of the subject will also recover.

Current devices to measure CFFF are expensive to manufacture and are not readily available to ophthalmologists, optometrists, medical professionals, or others desiring to measure and monitor the CFFF of their patients. For example, the Neuro-Opthalmology Clinic at the John A. Moran Eye Center utilizes an expensive, custom built CFFF measurement device, which is large, must be plugged into an electrical socket to be used, and is not portable. This particular device is manufactured by the University of Iowa. As a result of these disadvantages, as well as others, it is estimated that less than twenty of these devices are currently in use. Other, less expensive CFFF devices that are currently available to the practitioner do not provide accurate or reproducible results and can be difficult to use with young or old patients or patients with diseases of the eye.

Other tests are used by physicians to quantify optic nerve function. These include tests of visual acuity (e.g., eye charts), contrast sensitivity, color discrimination, perimetry (testing of peripheral vision), and visually evoked potential (VEP). In general, physicians use a combination of these tests to assess optic nerve function. VEP is the most objective of these tests and is measured by having the patient view an alternating checkerboard pattern while electrical activity in visual parts of the brain are measured through electrodes placed on the patient's head. Although sensitive and specific, measuring the VEP requires specialized equipment and specifically trained personnel, making this test expensive and not widely used.

Thus, there is a need in the art for an apparatus for measuring the CFFF of subjects that provides accurate results, is inexpensive and can be mass produced, can be used by a wide range of patients of any age and health, and is portable.

### SUMMARY OF THE INVENTION

In accordance with the purpose(s) of this invention, as embodied and broadly described herein, this invention, in one aspect; relates to an apparatus for measuring the critical flicker fusion frequency (CFFF) of a subject. In one aspect, the apparatus comprises at least two light sources, each configured to flicker at a predetermined frequency. In a further aspect, the apparatus comprises means for receiving a selection of one of the light sources, the selection indicating the light source that appears to the subject as fused (i.e., non-flickering). The apparatus also comprises, in one aspect, means for controlling the frequency of each of the light sources, the means being configured to adjust the predetermined frequencies at least in part in response to the selection made by the subject.

In another aspect, the invention relates to a method for measuring the CFFF of a subject. In one aspect, the method comprises providing an apparatus to the subject comprising at least two light sources. The method, in a further aspect, comprises flickering the light sources at first predetermined frequencies and receiving a first selection from the subject of a first of the light sources, the first selection indicating the light source that appears as a fused light to the subject. In yet a further aspect, the method comprises receiving a second selection from the subject of a second of the light sources, the second selection indicating the light source that appears as a fused light to the subject. The method can also comprise determining the subject's CFFF based at least in part on the second selection.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several aspects of the invention and together with the description, serve to explain the principles of the invention.

FIG. 1A is a schematic diagram of an exemplary computing device, according to one aspect of the present invention.

FIG. 1B is a schematic diagram of an exemplary processing system, according to one aspect of the present invention.

FIG. 2 is a schematic diagram of an apparatus for measuring the CFFF of a subject, according to one aspect of the present invention.

FIG. 3A is a schematic diagram of an apparatus having a display for prompting a subject to make a first selection from an array of light sources flickering at different frequencies in the range of 1 to 45 Hz, according to one aspect of the present invention.

FIG. 3B is a schematic diagram of the apparatus of FIG. 2A displaying a prompt for the subject to make a second selection from the array of light sources flickering at different frequencies in a narrower range (e.g., 21-30 Hz) that centers around the subject's first selection.

FIG. 4 shows an exemplary apparatus having an alpha-numeric display that is prompting a subject to make a first selection from an array of light sources, according to one aspect of the present invention.

FIG. 5 shows the apparatus of FIG. 4 prompting the subject to make a second selection from the array of light sources, according to one aspect of the present invention.

FIG. 6 shows the apparatus of FIG. 4, displaying the frequency of the subject's second selection, according to one aspect of the present invention.

FIG. 7 shows the internal components of the apparatus of FIG. 4, according to one aspect of the present invention.

FIG. 8 is schematic diagram of an apparatus for measuring the CFFF of a subject comprising an occluder, according to one aspect of the present invention.

FIG. 9 is a flowchart illustrating a method of measuring critical flicker fusion frequency of a subject, according to one aspect of the present invention.

FIG. 10 is a graphical illustration of the critical flicker fusion frequencies of test subjects measured by an apparatus according to one aspect of the present invention, plotted against the CFFFs measured by the CFFF measurement device currently produced by the University of Iowa and showing the correlation coefficient between the two sets of data.

FIG. 11 is a graphical illustration of the mean frequencies measured by both the apparatus according to one aspect of the present invention and the device currently produced by the University of Iowa, plotted against the differences between the frequencies as measured by both devices and showing the number of data points falling with ±2 standard deviations of the mean difference.

FIG. 12 is a graphical illustration of the critical flicker fusion frequencies of test subjects measured by an apparatus according to one aspect of the present invention, plotted against the VEP latency of the same test subjects.

FIG. 13 is graphical illustration of the critical flicker fusion frequencies of test subjects measured by the device currently produced by the University of Iowa, plotted against the VEP latency of the same test subjects.

FIG. 14 is a graphical illustration of the correlation of the data shown in FIGS. 12 and 13, combined.

FIG. 15 is a graphical illustration of the agreement between CFFF measurements measured by the device currently produced by the University of Iowa and CFFF measurements measured by an apparatus according to one aspect of the present invention, for a subset of 10 test subjects that also underwent VEP testing.

### DESCRIPTION OF THE PREFERRED EMBODIMENTS

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

As used in the specification and the appended claims, the singular forms “a,” “an” and “the” include plural referents unless the context clearly dictates otherwise. Thus, for example, reference to a “light source” includes can include two or more such light sources unless the context indicates otherwise.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

As used herein, the terms “optional” or “optionally” mean that the subsequently described event or circumstance can or can not occur, and that the description includes instances where said event or circumstance occurs and instances where it does not.

Various aspects of the present invention are described below with reference to block diagrams and flowchart illustrations of methods, apparatuses, and systems. It will be understood that each block of the block diagrams and flowchart illustrations, and combinations of blocks in the block diagrams and flowchart illustrations, respectively, can be implemented by computer program instructions. These computer program instructions can be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions can also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions can also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

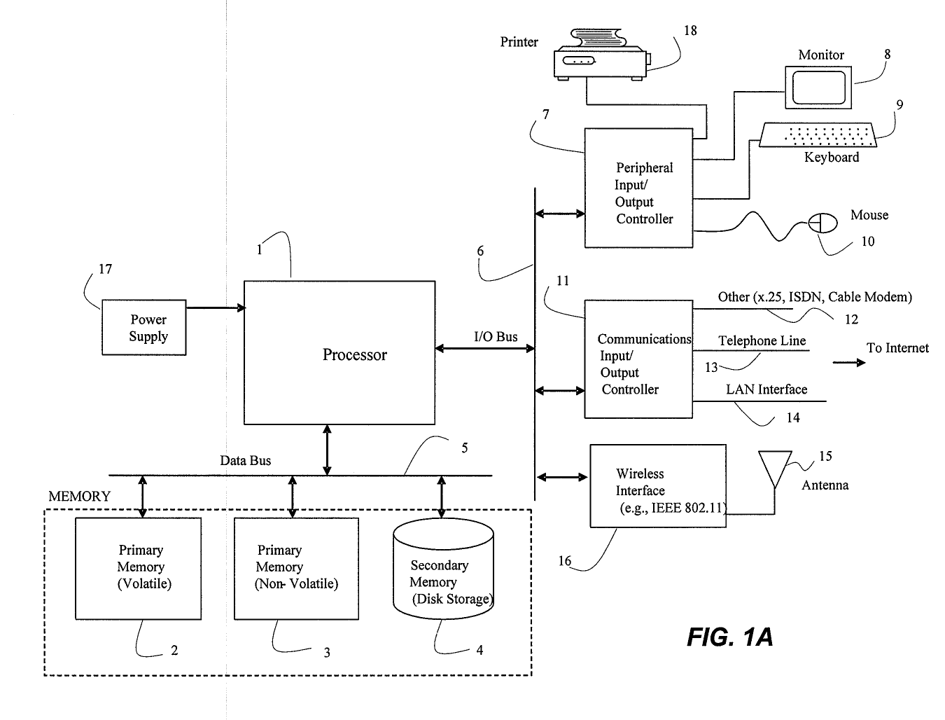

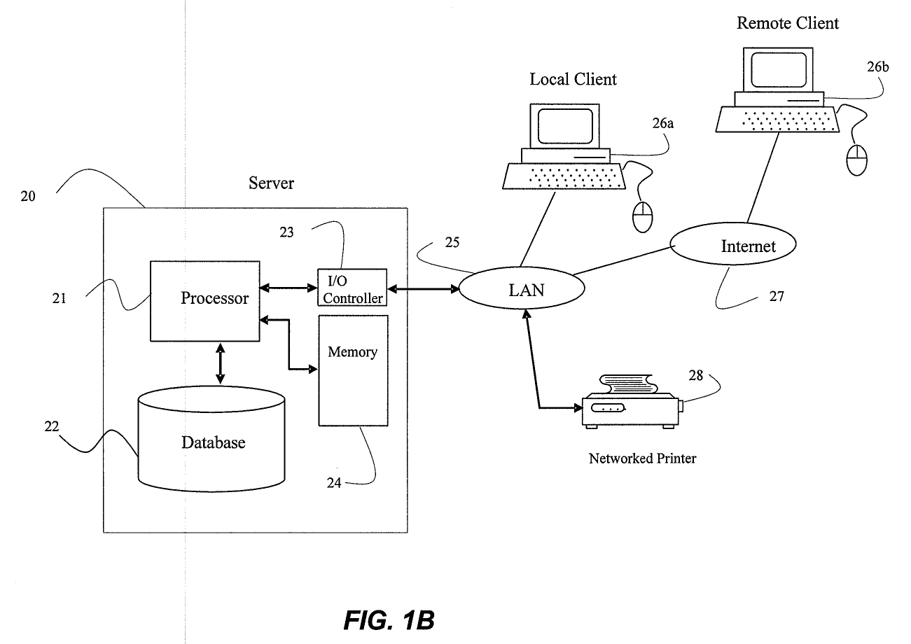

In the various aspects referenced herein, a “computer” or “computing device” can be referenced. Such computer can be, for example, a mainframe, desktop, notebook or laptop, a handheld device such as a data acquisition and storage device, or it can be a processing device embodied within another apparatus such as, for example, a set top box for a television system or a wireless telephone. In some instances the computer can be a “dumb” terminal used to access data or processors over a network. Turning to FIG. 1A, one aspect of a computing device is illustrated that can be used to practice aspects of the present invention. In FIG. 1A, a processor 1, such as a microprocessor, is used to execute software instructions for carrying out the defined steps. The processor receives power from a power supply 17 that also provides power to the other components as necessary. The processor 1 communicates using a data bus 5 that may be 4, 8, 16 or 32 bits wide (e.g., in parallel), for example. The data bus 5 is used to convey data and program instructions, typically, between the processor and memory. In the present aspect, memory can be considered primary memory 2 that is RAM or other forms which retain the contents only during operation, or it can be non-volatile 3, such as ROM, EPROM, EEPROM, FLASH, or other types of memory that retain the memory contents at all times. The memory could also be secondary memory 4, such as disk storage, that stores large amount of data. In some aspects, the disk storage can communicate with the processor using an I/O bus 6 instead or a dedicated bus (not shown). The secondary memory can be a floppy disk, hard disk, compact disk, DVD, or any other type of mass storage type known to those skilled in the computer arts.

The processor 1 also communicates with various peripherals or external devices using an I/o bus 6. In the present aspect, a peripheral I/o controller 7 is used to provide standard interfaces, such as RS-232, RS422, DIN, USB, or other interfaces as appropriate to interface various input/output devices. Typical input/output devices include local printers 18, a monitor 8, a keyboard 9, and a mouse 10 or other typical pointing devices (e.g., rollerball, trackpad, joystick, etc.).

The processor 1 typically also communicates using a communications I/O controller 11 with external communication networks, and can use a variety of interfaces such as data communication oriented protocols 12 such as X.25, ISDN, DSL, cable modems, etc. The communications controller 11 can also incorporate a modem (not shown) for interfacing and communicating with a standard telephone line 13. Finally, the communications I/O controller can incorporate an Ethernet interface 14 for communicating over a LAN. Any of these interfaces can be used to access a wide area network such as the Internet, intranets, LANs, or other data communication facilities.

Finally, the processor 1 can communicate with a wireless interface 16 that is operatively connected to an antenna 15 for communicating wirelessly with another device, using for example, one of the IEEE 802.11 protocols, 802.15.4 protocol, or a standard 3G wireless telecommunications protocols, such as CDMA2000 1x EV-DO, GPRS, W-CDMA, or other protocol.

An alternative aspect of a processing system that can be used is shown in FIG. 1B. In this aspect, a distributed communication and processing architecture is shown involving a server 20 communicating with either a local client computer 26a or a remote client computer 26b. The server 20 typically comprises a processor 21 that communicates with a database 22, which can be viewed as a form of secondary memory, as well as primary memory 24. The processor also communicates with external devices using an I/o controller 23 that typically interfaces with a LAN 25. The LAN can provide local connectivity to a networked printer 28 and the local client computer 26a. These can be located in the same facility as the server, though not necessarily in the same room. Communication with remote devices typically is accomplished by routing data from the LAN 25 over a communications facility to a wide area network 27, such as the Internet. A remote client computer 26b can execute a web browser, so that the remote client 26b can interact with the server as required by transmitted data through the wide area network 27; over the LAN 25, and to the server 20.

Those skilled in the art of data networking will realize that many other alternatives and architectures are possible and can be used to practice the preferred aspects. The aspects illustrated in FIGS. 1A and 1B can be modified in different ways and be within the scope of the present invention as claimed.

Reference will now be made in detail to the present preferred aspect(s) of the invention, examples of which are illustrated in the accompanying drawings. Wherever possible, the same reference numbers are used throughout the drawings to refer to the same or like parts.

The critical flicker fusion frequency of a subject can be a good indicator of the optic nerve function of the subject. In one aspect of the present invention, an apparatus is provided for measuring the critical flicker fusion frequency (CFFF) of a subject. In a further aspect, the apparatus comprises at least two light sources, which are each configured to flicker at a predetermined frequency. In one aspect, the predetermined frequency of at least one light source differs from the predetermined frequency of at least one other light source. The apparatus also comprises means for receiving a selection of one of the light sources from the subject. Because the apparatus is designed to measure the CFFF of the subject, it is contemplated that, in one aspect, the selection indicates the light source that appears to the subject as a fused (i.e., non-flickering) light. The apparatus can also comprise means for controlling the frequencies of each of the light sources.

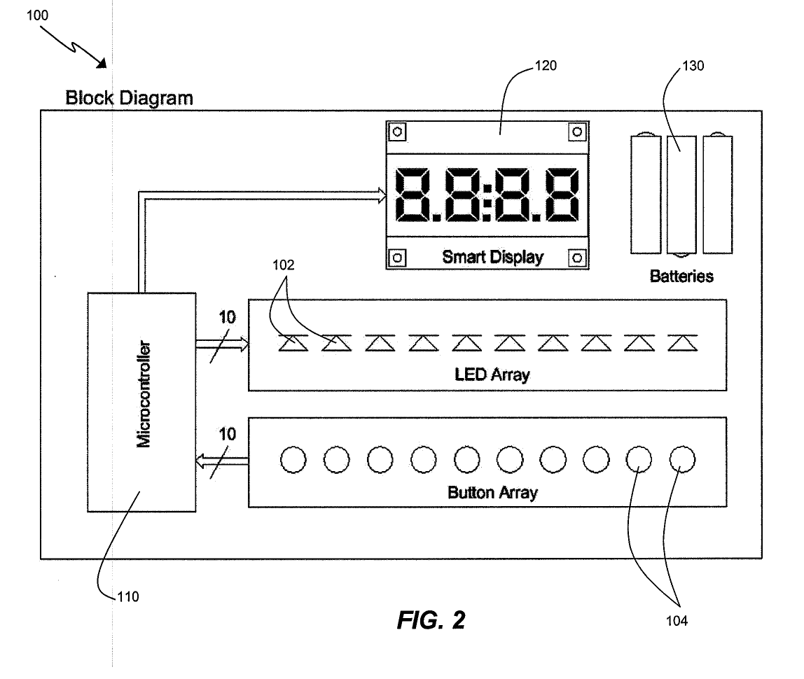

With reference to FIG. 2, in one aspect the light sources can be light emitting diodes (LEDs). The LEDs 102 can be arranged in an array, such as a linear array as shown in FIG. 2. Optionally, the light sources can be arranged in a non-linear array, such as, and without limitation, a circular array, rectangular array, a non-linear patterned array, or can be arranged randomly. When arranged in a linear array, the light sources can flicker at different predetermined frequencies that ascend from left to right, as viewed by the subject. Optionally, the frequencies can descend from left to right, as viewed by the subject. In an exemplary aspect, the apparatus 100 comprises from about two to about fifty light sources. Optionally, the apparatus comprises from about two to about thirty light sources. In other aspects the apparatus comprises from about two to about twenty light sources. In a particular aspect, such as that shown in FIG. 2, the apparatus comprises ten light sources.

In one aspect, the apparatus also comprises means for receiving a selection of a light source from the subject. In one aspect, the apparatus comprises one or more response buttons configured to receive a selection of at least one of the light sources from the subject. In a particular aspect, the apparatus comprises one response button for each light source. Thus, as shown in FIG. 2, in one aspect the apparatus 100 comprises ten LEDs 102 and ten response buttons 104, of which each response button corresponds to a respective LED. The apparatus can also comprise a microcomputer (or other computing device) such as, but not limited to, a microcontroller 110, that is configured for controlling all or most of the functions of the apparatus. For example, the microcontroller can be configured to control and adjust the frequencies of each light source. The microcontroller can also be configured to receive a selection of any of the response buttons and determine the LED to which the response button corresponds. In one aspect, a clock speed for the microcontroller can be selected to make predetermined values, such as selected integer values between 0 and 50 Hz (although other ranges are contemplated as being within the scope of this invention), possible while minimizing the amount of error for each value. In one exemplary aspect, the clock speed of the microcontroller 110 can be 9.8304, although it is contemplated that other clock speeds, such as but not limited to 10 MHz and 15 MHz, are within the scope of the present invention. The microcontroller can display the frequency of any of the LEDs or other information on a display 120, which can be a liquid crystal display (LCD). In one aspect, the display is an alphanumeric display.

The apparatus can be powered by batteries 130, an AC/DC power source, an other power supply, or any combination thereof. In one aspect, a constant current source or voltage regulator can be provided to power the light sources. In another aspect, a battery monitor can be provided to monitor battery level. It is contemplated that the apparatus, in various aspects, will be sized and powered so as to be portable. For example, if a conventional AC/DC power source is provided, the apparatus can be used in any environment having a conventional power outlet. The use of batteries allows the apparatus to be used anywhere.

As described above, in one aspect, the frequency of one light source differs from the frequency of at least one other light source. For example, the apparatus can comprise an array of light sources, each flickering at a unique predetermined frequency. Thus, the frequencies of the array of light sources define a first range of frequencies. The predetermined frequencies can be controlled and/or adjusted (such as by the microcontroller), to define second, third, etc., subset frequency ranges.

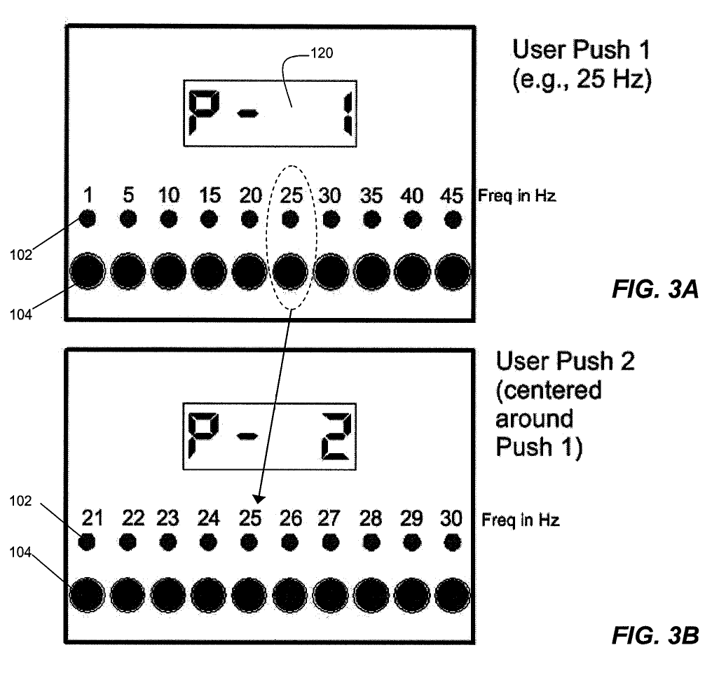

For example, with reference to FIG. 3A, a first array of light sources can be presented to the subject with ascending frequencies (from left to right, as viewed by the subject). The frequencies are shown in the figure for exemplary purposes only and, in various aspects, would not be visible to the subject using the apparatus. The first array of light sources can comprise ten LEDs flickering at frequencies ranging from 1 Hz to 45 Hz in increments of about 5 Hz. In other aspects, the range can be broader or narrower, and the frequency increments can be more or less than 5 Hz. For example, the frequency increments between light sources in an array can be from about 2 to about 5 Hz, including the increments of 2, 2.5, 3, 3.5, 4, 4.5, and 5 Hz. In other aspects, the frequency increments can be from about 0.5 to about 3 Hz, including the increments of 0.5, 1, 1.5, 2, 2.5 and 3 Hz. It is, of course, contemplated that any desired frequency range can be utilized for the selected predetermined frequencies.

The CFFF of a normal test subject (i.e., a test subject with a normal optic nerve function) typically falls in the range of 25 to 35 Hz. Thus, in one exemplary aspect and not meant to be limiting, the array of light sources defines a first frequency range that comprises the range of 25 to 35 Hz, such as a range of about 20 to about 40 Hz. Optionally, the range can be from about 10 to about 50 Hz. In another aspect, the range can be from 1 Hz to 45 Hz, as illustrated in FIG. 3A. However, if the subject is known to have a particular condition that alters the normal range of the patient's CFFF, the initial range of presented predetermined frequencies can be selected by the operator. For example, if a patient has a condition that would lower their CFFF, the initial range can be set lower, such as 20 to 30 Hz, 15 to 30 Hz, or other range.

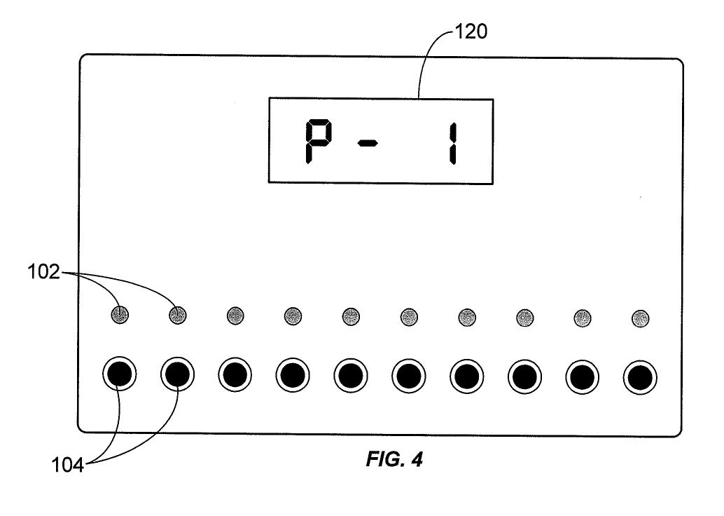

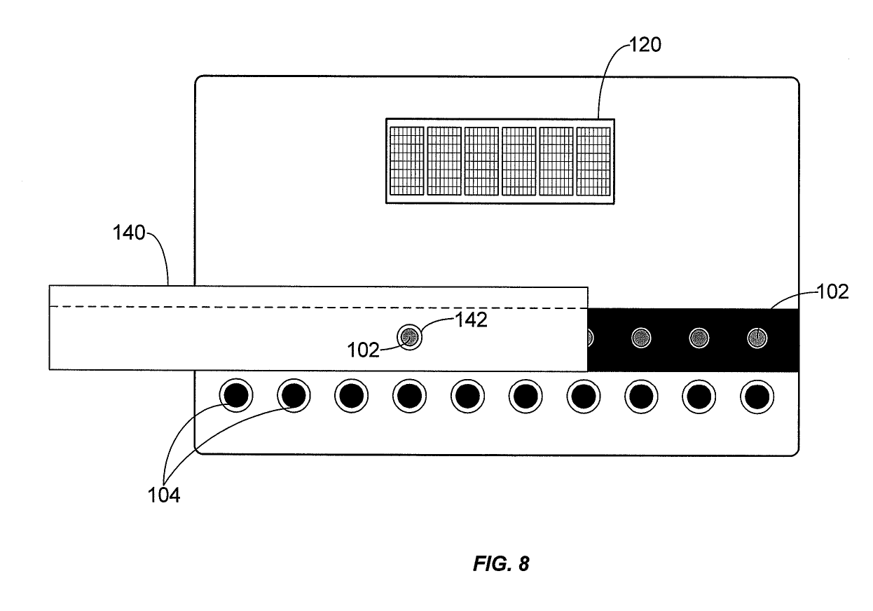

The apparatus in one aspect is configured to display information to the subject and physician or other operator of the apparatus. As shown in FIGS. 3A and 4, the display 120 can display “P-1” to prompt the subject to make a first selection. As described above, the subject can be instructed by the operator to select the LED of lowest frequency that appears to the subject as a fused light. For example, if the LEDs are presented in an array of ascending frequencies (from left to right as viewed by the subject), the subject can begin viewing the LEDs at the left hand side of the apparatus and view the LEDs from left to right until a light source appears as a fused light. In one aspect, an occluder is provided that is configured for blocking one or more light sources of the array of light sources. For example, the occluder can be configured to block all but one light source at a time, so that the subject can view each light source independently of the other light sources. The occluder can be slidably mounted on a top surface of the apparatus housing and is slidably moveable along the upper surface to block one or more of the light sources. In this manner, the subject can go back and forth between light sources if he is having difficulty determining which light source appears fused. The subject, thus, can also use the occluder to start viewing the light source having the highest frequency (such as at the right hand side of the apparatus as shown in FIG. 3A) and then move the occluder from right to left in order to identify the first light source that appears as flickering (or the last light source that appears as fused). FIG. 8 illustrates an apparatus according to one aspect of the present invention having a sliding occluder 140. As can be seen, the occluder has an aperture 142 through which a light source is visible to the subject. Although in FIG. 8 several light sources are visible other than through the aperture 142, it is contemplated that in one embodiment the occluder is of a length selected to cover all but one light source. Thus, even when alight source 102 at either end of the linear array is being viewed, all other light sources will be blocked from view of the subject by means of the occluder. In an alternative embodiment, it is contemplated that the occluder can be configured to block at least one of the lights of the array of lights during operation.

Optionally, as shown in FIG. 8, an occluder may be provided that is configured to block at least one light, such as a light that is perceived by the subject as flickering, but is configured to allow one or more lights to remain unblocked. For example, in an aspect in which a linear array having ascending frequencies from left to right is presented to the subject, the occluder may be configured to progressively block light sources that appear to the user as flickering (i.e., those that may no longer be under scrutiny by the subject). A subject may begin by viewing the left-most light source of the array, and determine that it is flickering. In this exemplary aspect, some of the light sources to the right of that light source may also appear as flickering, and some may appear as fused (such as, but not limited to, those toward the right-hand side of the apparatus). As the subject moves the occluder from left to right, the occluder will progressively block the light sources that the subject has determined appear as blinking (such as, but not limited to, those toward the left-hand side of the apparatus). At some point, such as when the subject has selected the first light source of the lowest frequency that appears as fused, all of the light sources that appear as flickering (if any) may be blocked by the occluder, while some of the light sources of higher frequency than the selected frequency that appear as fused (if any) may be visible to the subject. In one aspect, by progressively blocking the lights that appear to the subject as flickering, the occluder is configured to make it easier for the subject to distinguish between flickering and fused light sources by not simultaneously presenting (and possibly overwhelming) the eyes with light sources of which some appear as flickering and some appear as fused.

Regardless of the manner in which the subject is viewing the light sources, the subject can indicate the fused light source of lowest frequency by pressing a corresponding response button. In the example shown in FIG. 3A, the subject can identify the light source flickering at 25 Hz as the first light source in the ascending array that is fused. The subject can then push the response button under the LED that is flickering at 25 Hz to select this light. The microcontroller 110 is configured to receive this selection, which is indicative of a first estimate of the patient's CFFF. In one aspect, the microcontroller can display the selected frequency on the alphanumeric display 120. Optionally, or additionally, the microcontroller can adjust the frequencies of the LEDs to display an array of LEDs flickering in a second, narrower range as compared to the first range, such as 21 to 30 Hz, i.e., a subset of the first range of frequencies. It is contemplated that, in various aspects, the second range will comprise the frequency of the subject's first selection. In one aspect, the second range can center approximately around the frequency of the first selection. For example, as shown in FIGS. 3A and 3B, if the subject makes a first selection of 25 Hz, the second range of frequencies comprises the frequency of 25 Hz, and centers approximately around this value by defining a second range from 21 Hz to 30 Hz. Optionally, the second range can comprise the frequency of the first selection without centering this value.

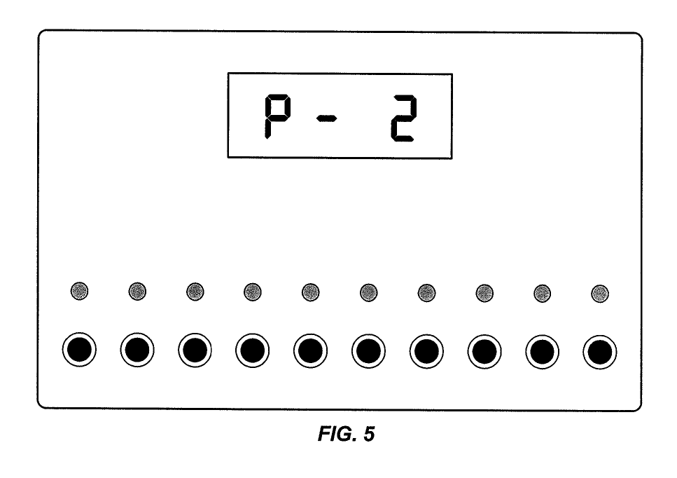

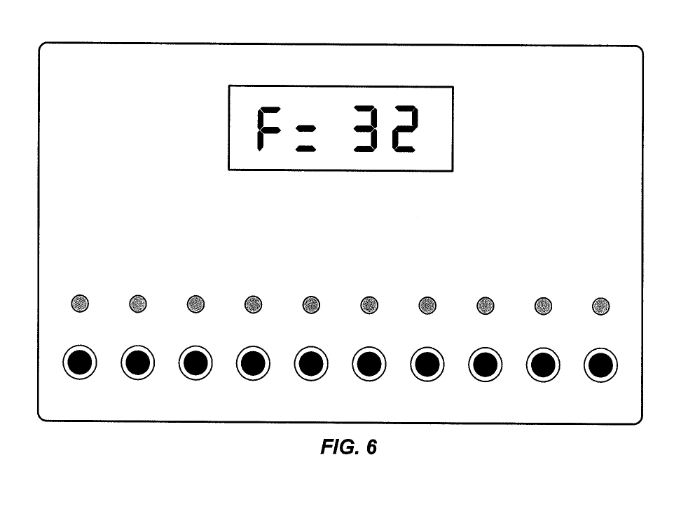

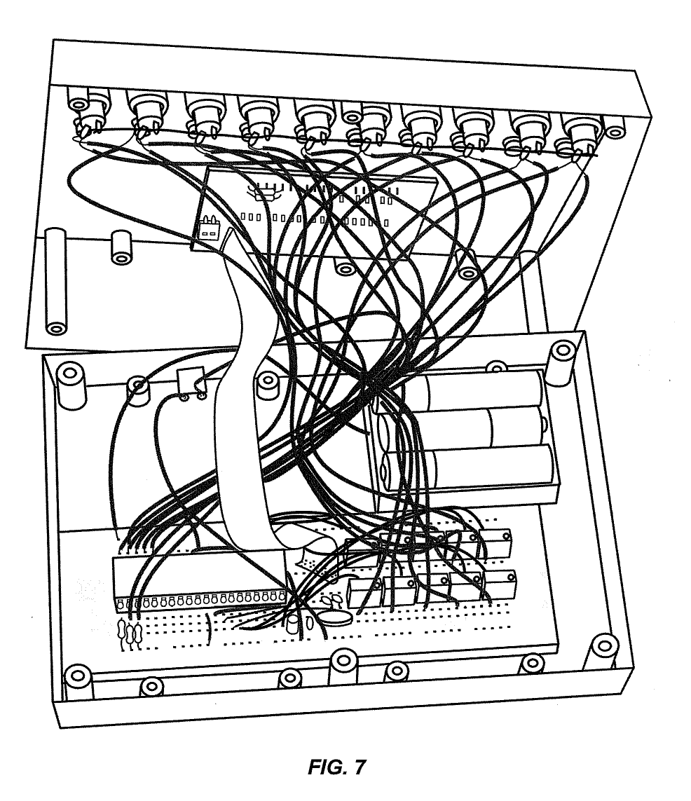

The subject can then prompted via the display (or by the operator), as shown in FIGS. 3B and 5, to make a second selection that represents the LED of lowest frequency that appears to the subject as a fused light. Similarly as described above, the subject can use an occluder to view one or a few light sources at a time, and can view the light sources in any order. The microcontroller is configured to receive this second selection, which is indicative of a second estimate of the patient's CFFF and is more accurate that the previously determined first estimate of the patient's CFFF. In one aspect, the second selection can be designated as the subject's critical flicker fusion frequency. This value can be displayed by the microcontroller on the alphanumeric display 120 so that it can be recorded by the operator, for example as shown in FIG. 6.

It is contemplated that the process described above can be repeated through several (i.e., two or more) iterations in order to determine the subject's CFFF. For example, the process can be repeated through three or more, rather than just one or two, iterations. In an apparatus having an array of ten lights, the first range of frequencies can range from 1-55 Hz in increments of about 6 Hz, including the frequencies of 1, 7, 13, 19, 25, 31, 37, 43, 49 and 55 Hz. For exemplary purposes only, the subject can make a first selection of the light source flickering at 19 Hz, which frequency corresponded to the lowest fusion flicker rate of the patient. Upon receipt of the subject's first selection, the microcontroller is configured to adjust the frequencies of the light sources to a narrower range that comprises a subset of the subject's first selection (e.g., 19 Hz). The second range can center around the frequency of 19 Hz and range from 4-31 Hz, in increments of about 3 Hz. Because, in this aspect, there is an even number of light sources, the second range can center around the frequency of 19 Hz and range from 7-34 Hz, in increments of about 3 Hz. The subject can make a second selection of a frequency of 22 Hz for example, which frequency corresponded to the lowest fusion flicker rate of the patient. Upon receiving the subject's second selection, the microcontroller is configured to adjust the frequencies of the light sources to a third, narrower subset range that comprises the subject's second selection (e.g., 22 Hz). The third range can center around 22 Hz and range from 20-24.5 Hz in increments of 0.5 Hz. Thus, with increasing iterations, it is possible to obtain a successively more accurate CFFF measurement. It is to be understood that the values provided in above were from exemplary purposes only, and each range can include any frequency values and the increments are not to be limited to those described above.

It is contemplated that, in one aspect, by using an iterative approach to determining a subject's CFFF, significant time savings can be achieved as compared to current methods of measuring CFFF. For example, current devices may require a subject to view a light source flickering at over fifty different frequencies in order to determine the subject's CFFF. This method of testing can not only be time consuming, but can also be difficult and tiring for a patient to complete. On the other hand, by using an apparatus such as described throughout with respect to various aspects, having a few light sources that represent a range of frequencies that can be quickly and successively narrowed by successive iterations, a subject can quickly and accurately complete the test.

In other aspects, the apparatus can comprise means for calibrating the brightness of each of the light sources prior to viewing by the subject. For instance, the light sources can be calibrated so that the brightness is uniform across all of the light sources. The light sources can be calibrated prior to shipping the apparatus to its intended user or operator. Optionally, the light sources can be calibrated by the operator prior to providing the apparatus to the subject for viewing.

The apparatus can also comprise means for measuring ambient light. Generally, accurate results (i.e., measurements of a subject's CFFF) can be obtained regardless of the amount of light surrounding the subject. However, it can be useful to measure the ambient light so that the apparatus can be used in a consistent operating environment. The apparatus can also comprise means for adjusting the brightness of the light sources in response to the ambient light measurement. This also can be helpful in maintaining a consistent operating environment.

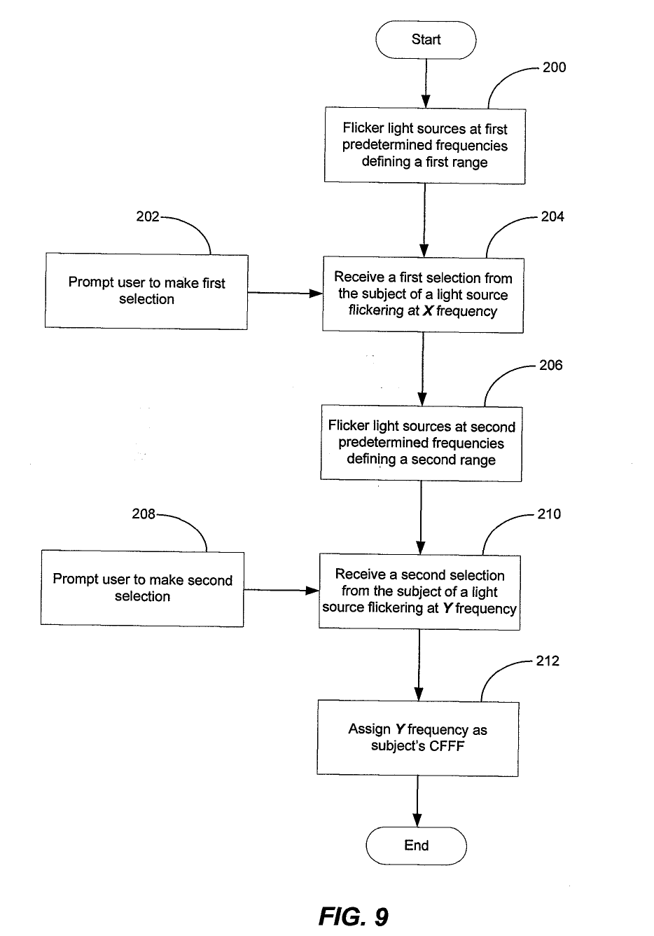

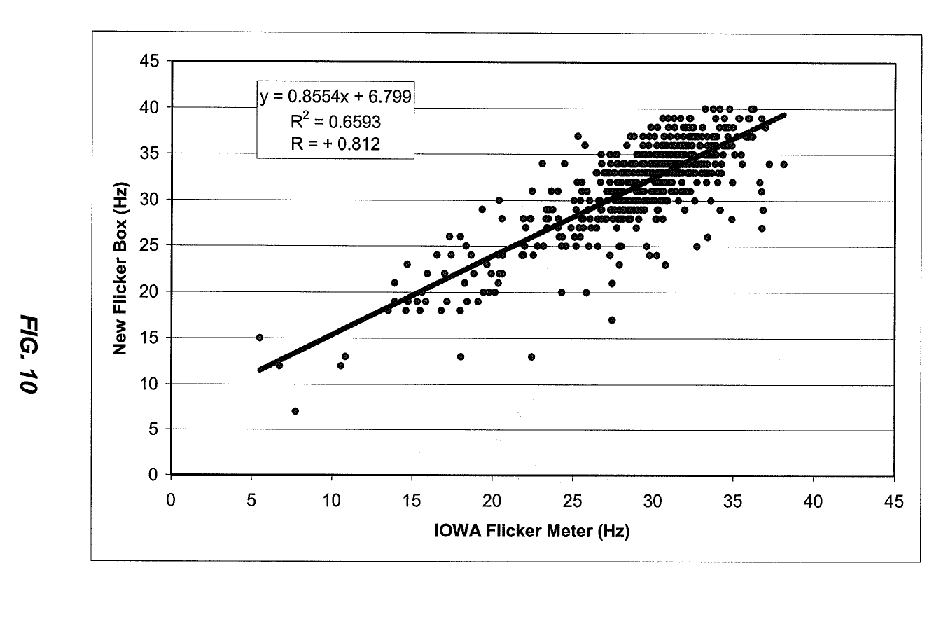

In another embodiment of the present invention, a method for measuring the critical flicker fusion frequency of a subject is provided. The method comprises, in one aspect, providing an apparatus to the subject having at least two light sources flickering at first predetermined frequencies. The apparatus can be any apparatus as described above with regard to one or more aspects of the present invention. With reference to FIG. 9, at step 200, the light sources are flickered at first predetermined frequencies. As described above, each light source can flicker at a unique predetermined frequency. These first predetermined frequencies, thus, can define a first range of frequencies.

At step 202, the subject can be prompted to make a first selection. The subject can be prompted by the operator (such as the subject's physician, ophthalmologist, medical professional, or other such operator), or can be prompted via a display on the apparatus. Step 204 comprises receiving a first selection from the subject. The first selection can indicate, for example, the light source that appears as a fused light to the subject. As described above, in one aspect, the light sources are presented in a linear array with ascending frequencies (from left to right as viewed by the subject). The first selection can indicate the light source of lowest frequency (i.e., the left-most light source) that appears as fused to the subject. The frequency value of the subject's first selection can be displayed on the apparatus display.

After receiving the subject's first selection, at step 206 the light sources are flickered at second predetermined frequencies. Each of the second predetermined frequencies can be unique and can define a second range. In one aspect, the second range is a narrower subset of the first range. In another aspect, the second range comprises the frequency of the subject's first selection. For example, the second range can center approximately around the frequency of the subject's first selection.

At step 208, the subject can be prompted to make a second selection. The second selection can indicate the light source that appears as a fused light to the subject, such as described above. Step 210 comprises receiving the second selection from the subject. In one aspect, the frequency of the second selection can be the same as the frequency of the first selection. Optionally, the frequency of the second selection can be higher or lower than the frequency of the first selection. The frequency of the second selection can be displayed on an alpha-numeric display of the apparatus. This can allow the operator, for example, to record the frequency value of the second selection. In one aspect, the subject's CFFF is determined based at least in part on the second selection at step 212. In a particular aspect, the frequency value of the second selection is assigned as the subject's CFFF. Optionally, additional iterations can be performed to determine the subject's CFFF.

In various aspects, an occluder is provided that is configured to block at least one of the light sources from view of the subject. In a particular aspect, the occluder can be configured to block all but one light source from view of the subject. In one exemplary aspect, the occluder can be slidably mounted on an upper surface of the apparatus housing such that it is selectably movable along the housing. In this manner, a subject can view the light sources in any order, and can go back and forth between two or more light sources to determine which light source appears fused. In one aspect, the subject's eyes can be tested separately to determine the CFFF of each of the subject's eyes. Thus, the subject can be asked to close one eye, block one eye with his or her hand, etc. in order to test each eye individually. The CFFF measuring process as described throughout can be repeated for the subject's other eye.

In other aspects, the light sources can be calibrated prior to providing the apparatus to the subject for viewing. For example, the brightness of each light source can be calibrated so that all of the light sources have the same brightness. The ambient light can also be measured so that the process of measuring multiple subjects' CFFFs can be performed in consistent operating environments. The brightness of the light sources can be adjusted at least partially in response to the ambient light measurement.

### EXPERIMENTAL

The following examples are put forth so as to provide those of ordinary skill in the art with a complete disclosure and description of how the apparatuses, systems and/or methods claimed herein are made and evaluated, and are intended to be purely exemplary of the invention and are not intended to limit the scope of what the inventors regard as their invention. Efforts have been made to ensure accuracy with respect to numbers (e.g., frequency measurements, etc.), but some errors and deviations should be accounted for.

### Example 1

### Flicker Box vs. Iowa Flicker Meter

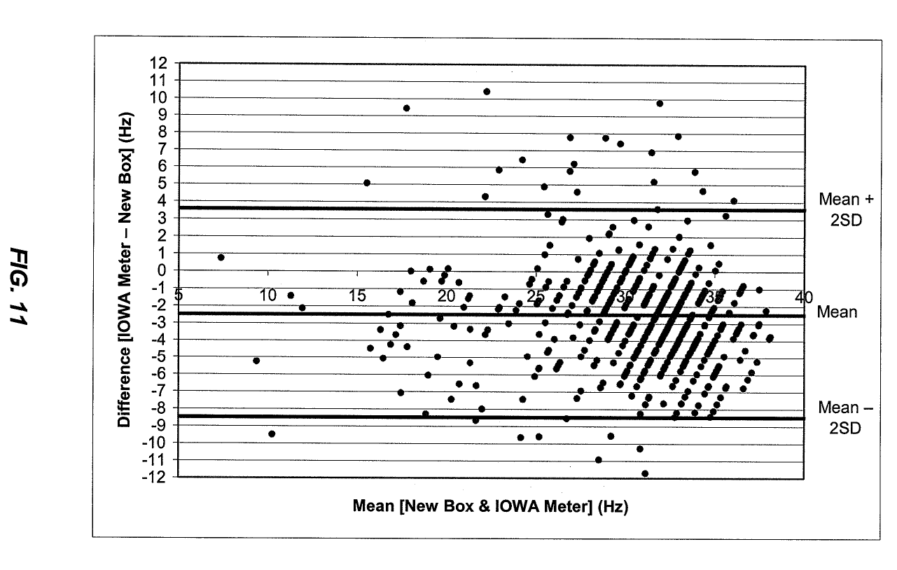

A test was conducted to measure critical flicker fusion frequencies of 500 eyes from 250 patients using both an exemplary apparatus of the present invention (referred to hereinafter as the “Flicker Box”, for exemplary purposes only) comprising aspects of the present invention and the device currently produced by the University of Iowa (referred to hereinafter as the “Iowa Flicker Meter”). FIG. 10 is a graphical representation of the results of the test. The data shown have a correlation coefficient (R²) of +0.66, which indicates a strong correlation between the results of the measurements taken by the Iowa Flicker Meter and the Flicker Box.

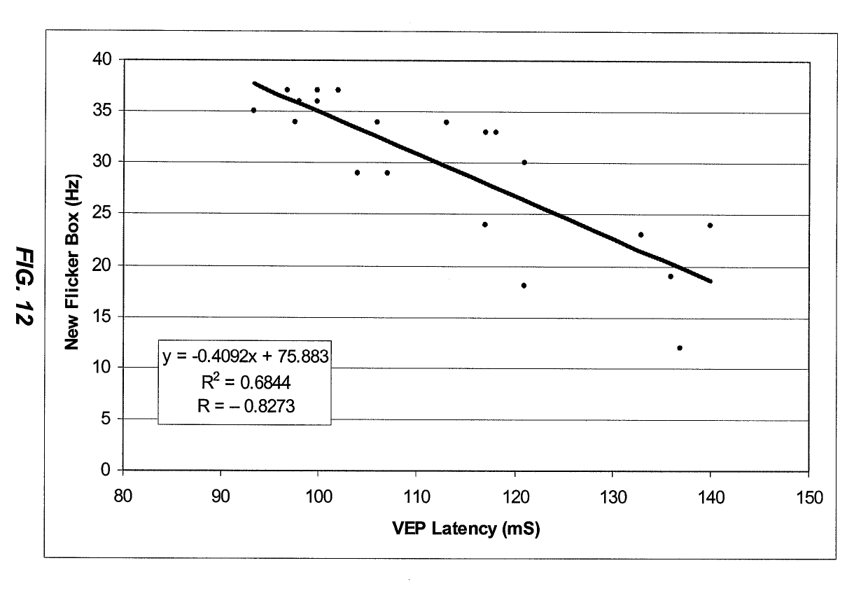

FIG. 11 is a graphical illustration, using the same test data, of the mean frequencies (measured by both the Iowa Flicker Meter and the Flicker Box) plotted against the difference in CFFF values measured by both devices. On average, CFFF measurements measured by the Flicker Box were 2.5 Hz above those measured using the Iowa Flicker Meter. Sixty-eight percent of CFFF measurements measured by the Flicker Box and the Iowa Flicker Meter were within +/−3 Hz of each other. Ninety-five percent of CFFF measurements measured by the Flicker Box and the Iowa Flicker Meter were within +/−6 Hz of each other.

### Example 2

### Flicker Box vs. Visually Evoked Potential (VEP)

As described above, one way to measure optic nerve function of a subject is to test the subject's visually evoked potential (VEP) using an objective, electrophysiologic test using a VEP instrument that is sensitive and specific. Due to the VEP's sensitivity and specificity, the VEP is often considered the “gold standard” of tests to quantify a subject's optic nerve function. However, as discussed above, measuring VEP requires specialized equipment, specially trained personnel, and is a time consuming test. The CFFF is a suitable alternative indication of optic nerve function.

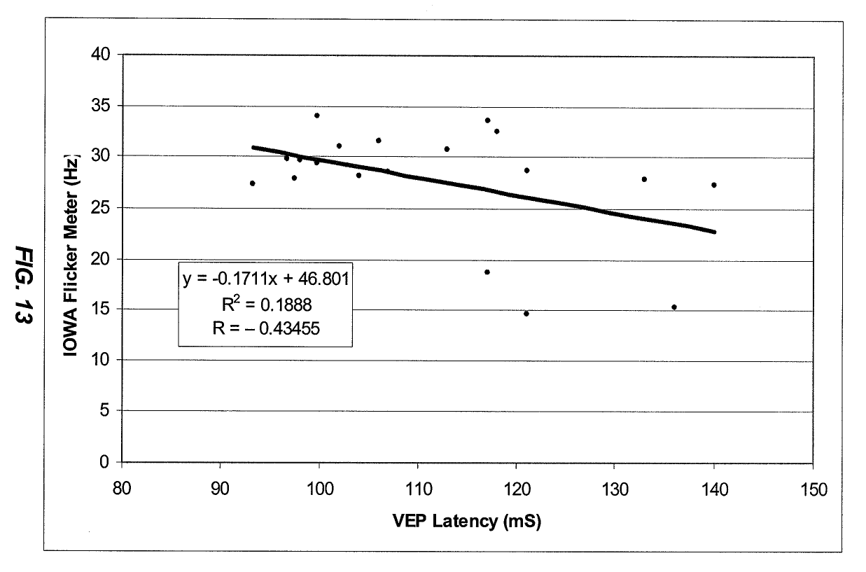

A test was conducted on a subset of ten patients from the pool of 250 patients (discussed above with regard to Example 1) to determine if there was a correlation between CFFF measurements (as measured by both the Flicker Box and the Iowa Flicker Meter) and VEP latency. VEP latency is the time lag between the presentation of a visual stimulus and the detection of an electrical signal in the visual center of the brain. Twenty eyes from ten patients were tested. FIG. 12 is a graphical illustration of the results of the data of this test. As can be seen, the correlation between CFFF, as measured by the Flicker Box, and VEP latency was very high (R²=0.68).

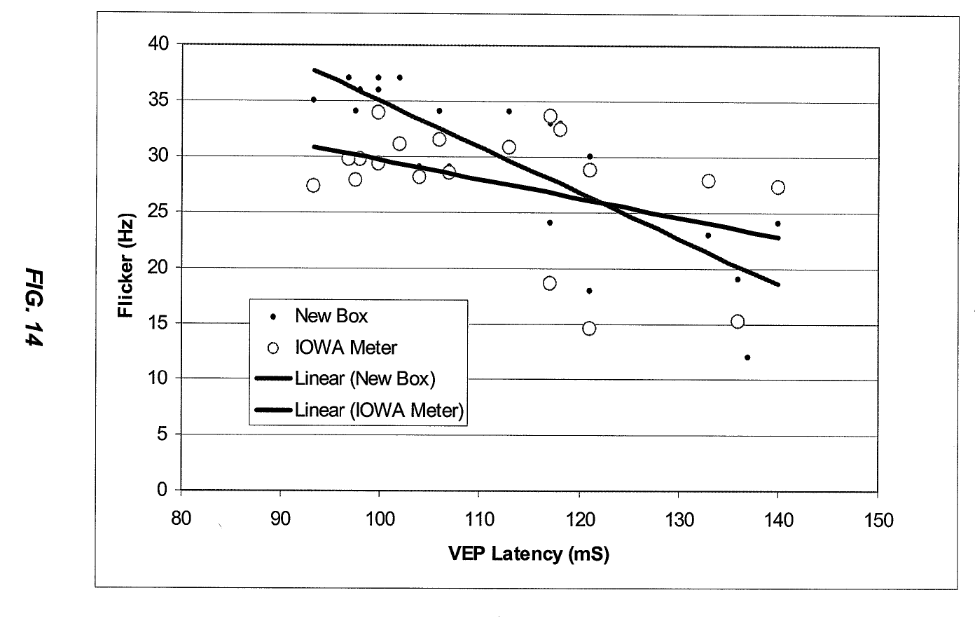

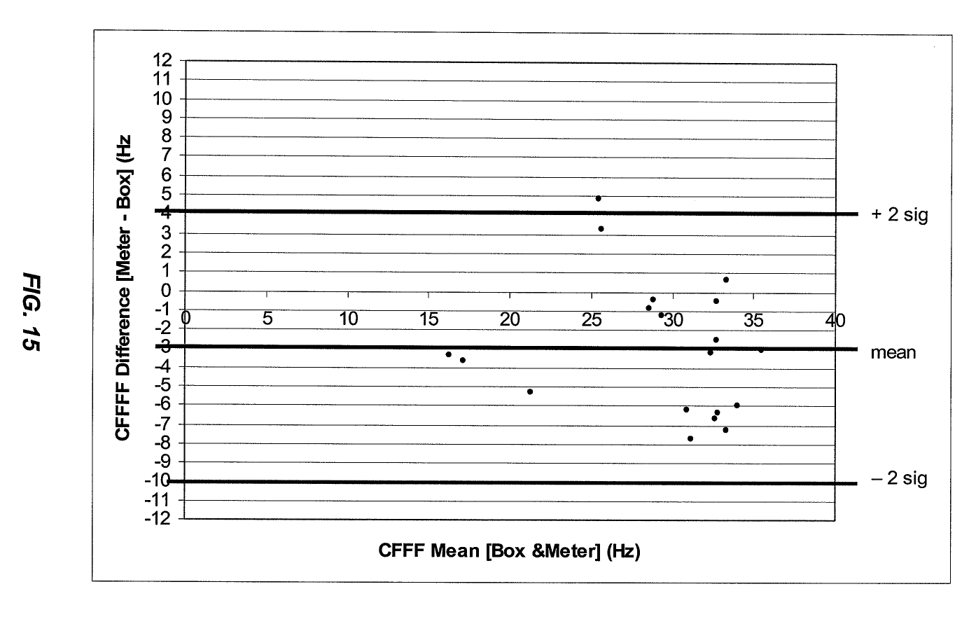

The correlation between CFFF, as measured by the Iowa Flicker Meter, and VEP latency was not nearly as high (R²=0.19). The results of this test are shown in FIG. 13. FIG. 14 compares the correlations obtained for CFFF measured by the Flicker Box and the CFFF measured by the Iowa Flicker Meter with VEP latency. These are the same data from FIGS. 12 and 13, combined. These results demonstrate that CFFF as measured by the Flicker Box is more strongly correlated with VEP latency than CFFF as measured by the Iowa Flicker Meter. FIG. 15 is a graphical illustration of the agreement between the CFFF measurements taken by the Flicker Box and the Iowa Flicker Meter for this subset of ten subjects.

Taking into account the results described in Examples 1 and 2, above, an apparatus according to one aspect of the present invention is capable of producing results of high accuracy as compared with currently used (and highly specialized and expensive) instruments. Additionally, an apparatus according to aspects of the present invention can be capable of providing faster results, can be easier for a patient to use, and can be less expensive to produce, thus making it available for use by a greater number of physicians than the currently known devices and instruments.

Although the previous description of aspects of the present invention focused on using CFFF measurements as an indication of optic nerve function, it is contemplated that CFFF measurements can be useful indications of other physical or psychophysical conditions not necessarily related to opthalmology. For example, CFFF can also be a psychophysical measure of alertness and concentration. This may be useful in industries in which a subject's job and/or responsibilities require the subject to have a certain level of alertness, such as truck drivers, airline and armed forces pilots, medical doctors (such as residents who typically work in very long shifts), and factory workers. It may be used to measure a patient's recovery from anesthesia. In other aspects, CFFF may be a measure of intoxication in a subject. Both anesthesia and intoxicants are known to slow conduction through a subject's nerves, thus resulting in a lower than normal CFFF measurement. It is also contemplated that CFFF can be used as a cognitive indicator in medical drug studies, such as those involving antidepressant medication, or testing the effectiveness of migraine treatment. CFFF may also be an early indicator of Alzheimer's Disease and other diseases affecting the brain.

Various publications were referenced in preparation of this application. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

(1) Brenton, R. S.; Thomson, H. S.; & Maxner, C., New Methods of Sensory Visual Testing: “Critical Flicker Fusion Frequency: A New Look at an Old Test”, Chapter 3, pp. 29-52 (Wall, M. & Sadun, A. A., eds., Springer-Verlag 1989).
(2) Jacobson, D. M. & Olson, K. A., Impaired Critical Flicker Fusion Frequency in Recovered Optic Neuritis, Ann. Neurol. 1991:30:312-215.
(3) Salmi, T., Critical Flicker Fusion Frequencies in MS Patients with Normal or Abnormal Pattern VEP, Acta. Neurol. Scand., 1985:71:354-358.

What is claimed is:

1. An apparatus for measuring the critical flicker fusion frequency of a subject, comprising:
at least two light sources, wherein each of the at least two light sources is configured to flicker at a predetermined frequency;
means for receiving a first selection from the subject, the first selection indicating a light source selected from the at least two light sources that appears as a fused light source to the subject;
means for controlling the frequency of each of the at least two light sources, the means being configured to adjust the predetermined frequencies at least in part in response to the first selection.

2. The apparatus of claim 1, wherein the at least two light sources comprise light emitting diodes.

3. The apparatus of claim 1, wherein the at least two light sources comprise from about two to about fifty light sources.

4. The apparatus of claim 1, wherein the at least two light sources comprise from about two to about thirty light sources.

5. The apparatus of claim 1, wherein the at least two light sources comprise from about two to about twenty light sources.

6. The apparatus of claim 1, wherein the at least two light sources comprise ten light sources.

7. The apparatus of claim 1, wherein the predetermined frequency of a first of the at least two light sources differs from the predetermined from a second of the at least two light sources.

8. The apparatus of claim 7, wherein the difference in frequency is about 5 Hz.

9. The apparatus of claim 7, wherein the difference in frequency is approximately 2 Hz.

10. The apparatus of claim 7, wherein the difference in frequency is approximately 1 Hz.

11. The apparatus of claim 1, wherein the at least two light sources are configured in an array.

12. The apparatus of claim 1, wherein the at least two light sources are configured in an array of ascending frequencies.

13. The apparatus of claim 1, wherein the at least two light sources are configured in an array of descending frequencies.

14. The apparatus of claim 1, wherein the predetermined frequencies of the at least two light sources define a first range of frequencies.

15. The apparatus of claim 14, wherein in response to the first selection, the means of controlling the predetermined frequencies is configured to adjust the at least two light sources to flicker at second predetermined frequencies, the second predetermined frequencies defining a second range of frequencies.

16. The apparatus of claim 15, wherein the second range of frequencies is more narrow than the first range of frequencies.

17. The apparatus of claim 16, wherein the second range of frequencies comprises the frequency of the light source selected by the subject from the first range of frequencies.

18. The apparatus of claim 17, wherein the second range of frequencies centers around the frequency of the light source selected by the subject from the first range of frequencies.

19. The apparatus of claim 17, wherein the means for receiving a first selection from the subject is further configured to receive a second selection from the subject, the second selection indicating a light source selected from the at least two light sources that appears as a fused light source to the subject.

20. The apparatus of claim 19, wherein the second selection designates the subject's critical flicker fusion frequency.

21. The apparatus of claim 1, further comprising means for displaying the frequency of at least one of the at least two light sources.

22. The apparatus of claim 1, further comprising means for occluding at least one of the at least two light sources.

23. The apparatus of claim 22, further comprising a housing having an upper surface, wherein the at least two light sources are disposed on the upper surface, and wherein the means for occluding at least one of the at least two light sources comprises an occluder slidably mounted to at least a portion of the upper surface of the housing.

24. The apparatus of claim 23, wherein the occluder is slidably moveable along the upper surface of the housing.

25. The apparatus of claim 1, further comprising means for calibrating the brightness of each of the at least two light sources prior to viewing by the subject.

26. The apparatus for claim 1, further comprising means for measuring ambient light.

27. The apparatus of claim 26, further comprising means for adjusting the brightness of the at least two light sources in response to an ambient light measurement.

28. The apparatus of claim 1, wherein the means for receiving a first selection from the subject comprises at least one response button.

29. The apparatus of claim 28, further comprising at least two response buttons, each of the at least two response buttons corresponding to a respective light source of the at least two light sources.

30. The apparatus of claim 1, wherein the apparatus is powered by batteries.

31. The apparatus of claim 1, wherein the apparatus is portable.

32. A method for measuring the critical flicker fusion frequency of a subject, comprising:
providing an apparatus to the subject, comprising at least two light sources, wherein each of the at least two light sources is configured to flicker at a predetermined frequency;
flickering the at least two light sources at first predetermined frequencies;
receiving a first selection from the subject of a first of the at least two light sources, the first selection indicating a light source that appears as a fused light to the subject;
flickering the at least two light sources at second predetermined frequencies in response to the first selection;
receiving a second selection from the subject of a second of the at least two light sources, the second selection indicating a light source that appears as a fused light to the subject; and
determining the subject's critical flicker fusion frequency based at least in part on the second selection.

33. The method of claim 32, wherein the first predetermined frequencies define a first range of frequencies and the second predetermined frequencies define a second range of frequencies.

34. The method of claim 33, wherein the second range of frequencies is narrower than the first range of frequencies.

35. The method of claim 34, wherein the second range of frequencies comprises the frequency of the first light source.

36. The method of claim 32, further comprising displaying the first selection on an alpha-numeric display.

37. The method of claim 32, further comprising displaying the second selection on an alpha-numeric display.

38. The method of claim 32, further comprising providing an occluder configured to block at least one of the at least two light sources from view of the subject.

39. The method of claim 38, wherein the occluder is configured to block all but one of the at least two light sources from view of the subject.

40. The method of claim 38, wherein the apparatus further comprises a housing having an upper surface, wherein the at least two light sources are disposed on the upper surface, and wherein the occluder is slidably mounted to at least a portion of the upper surface of the housing.

41. The method of claim 40, wherein the occluder is slidably movable along the housing.

42. The method of claim 32, further comprising calibrating each of the at least two light sources prior to providing the apparatus to the subject.

43. The method of claim 32, further comprising measuring ambient light.

44. The method of claim 43, further comprising adjusting brightness of the at least two light sources at least partially in response to the ambient light measurement.
